(19) United States
(12) Patent Application Publication
(10) Pub. No.: US 2019/0000626 A1
(43) Pub. Date: Jan. 3, 2019

(54) PENILE PROSTHESIS

(71) Applicant: FIDELIS MEDICAL LTD., Tel Aviv (IL)

(72) Inventors: Michael G TAL, Savyon (IL); Assaf LEON, Petah Tikva (IL); Amit GREENER, Tel Aviv (IL); Sefi SHACHRUR, Pardes-Hana Karkur (IL); Eyal SANDACH, Yehud (IL)

(73) Assignee: Fidelis Medical Ltd., Tel Aviv (IL)

(21) Appl. No.: 16/064,595

(22) PCT Filed: Dec. 21, 2016
(86) PCT No.: PCT/IB2016/057892

§ 371 Date: Jun. 21, 2018

Related U.S. Application Data
(60) Provisional application No. 62/270,643, filed on Dec. 22, 2015.

(51) Int. Cl. A61F 2/26

(52) U.S. Cl. CPC A61F 2/26; A61F 2250/0003

(57) ABSTRACT

An inflatable penile prosthesis (10) that obtains a penile erection in response to axial forces applied thereon to achieve erection that simulates a natural erection process.

### FIELD OF THE INVENTION

The present invention relates, in some embodiments thereof, to an inflatable penile prosthesis which is easy to operate, imperceptible and achieves a penile erection that simulates the natural erection process.

### BACKGROUND

Erectile dysfunction (ED) or impotence is a sexual dysfunction characterized by the inability to develop or maintain an erection of the penis during sexual activity. In a male, dilation of the arteries leading to the corpus cavernosum of the penis causes engorgement with attendant enlargement and rigidity of the penis, while the enlargement constricts blood flow from the penis through venous channels. Various psychological and physiological causes (e.g., long term diabetes, damage to the spinal cord, neuropathy, atherosclerosis or damages after pelvic surgery) are associated with ED or impotence in the human male. Regardless the cause, ED or impotence has a tremendous effect on the quality of life and psychological state of a male subject.

Implantable penile prostheses are common in the treatment of ED or impotence. Penile implantable prostheses are overall divided to inflatable and non-inflatable prostheses. Of the inflatable prostheses, one most commonly used is a three-piece prosthesis that includes one or more cylinders, a liquid reservoir that communicates with the cylinder(s), and a pump employed to move liquid from the liquid reservoir into the cylinder(s). Although this prosthesis provides an efficient penile erection, operation is inherently difficult due to the lack of direct access to the prosthesis. In particular, the pump is manually actuated through the scrotum, and locating and operating the pump can be difficult and commonly causes discomfort to the patient. An added drawback is that the implantation of this prosthesis requires an extensive surgical procedure involving not only the positioning of the cylinders within the corpus cavernosum, but also the implantation of the pump and the reservoir within the abdomen and scrotum, respectively. The extensive surgical procedure for implanting this three pieces prosthesis can be a significant deterrent. Two-pieces and one piece penile prostheses are further available, but those prostheses are also associated with discomfort to the patient caused by manual pumping through the scrotum or through the distal end of the penile shaft to facilitate fluid transfer from a reservoir to an inner chamber.

Non-inflatable penile prostheses include, for example, malleable penile prostheses that are semi-rigid penile implants including two separate cylinders. These penis implants never change in size and maintain a certain position when not manipulated, but can be manipulated to bend or be straight. The malleable penile implant is generally maintained in a downward position, and bends into an upward position prior to sexual intercourse. The advantage of the malleable penile prosthesis is the technical ease of insertion, and ease of use. The disadvantages of the malleable penile implants include the semi-rigid appearance of the penis at all times, an increased risk of discomfort and erosion and that it is less soft when flaccid and less rigid when firm.

There is thus a need for an improved penile prosthesis, which simulates natural physiological erection, which is comfortable to a patient and which may be readily implanted within a penis.

### SUMMARY OF THE INVENTION

The present invention, relates, in some embodiments thereof, to a penile prosthesis that is implantable within the penis, and that includes an inflatable tubular member and a fluid reservoir. The penile prosthesis of the invention achieves a penile erection upon fluid flowing and inflating the inflatable tubular member. The present invention further relates to methods of using an inflatable penile prosthesis of the invention, for treating erectile dysfunction.

Advantageously, the penile prosthesis of the invention is operable to induce a penile erection in a manner that closely mimics the natural erection process. In this regard, a penile erection is effected in response to push and/or pull forces applied by a user on the penile prosthesis. This is in contrast with prior art inflatable penile prostheses in which the manual pumping action is that of squeezing, such as squeezing a bulb to pump fluid from the reservoir or squeezing or pinching a portion of a tube. Squeezing or pinching is disadvantageous because this action is contrary to the natural longitudinal, rubbing movement to cause an erection and can also hurt if done incorrectly. In the present invention, these problems are solved by the natural longitudinal (e.g., rubbing) movement to cause pumping.

Further advantageously, the penile prosthesis of the invention may comprise a reservoir, being minimal in size, allowing a prosthesis that can be entirely implanted within the penis and that can optionally be of one-piece. This particular penile prosthesis structure affords a much less complex surgical procedure involving implantation of the entire prosthesis within the penis.

According to an aspect of the present invention, there is provided a penile prosthesis for implantation within a penis of a subject in need thereof, the penile prosthesis comprising:

an inflatable tubular member extending from a proximal end and a distal end and defining a lumen; and

a fluid reservoir for holding fluid and in fluid communication with the inflatable tubular member;

wherein the penile prosthesis transforms from a flaccid state to an erect state upon fluid flow from the reservoir to the lumen of the inflatable tubular member, and wherein the transformation is actuated via at least one of: push forces, and pull forces, applied on the penile prosthesis.

According to an aspect of the invention there is provided a penile prosthesis for implantation within the penis of a subject in need thereof, the penile prosthesis including an inflatable tubular member defining a lumen; a source of pressurizing fluid; a fluid reservoir for holding fluid and in fluid communication with the source of pressurizing fluid and the inflatable tubular member; and actuating mean(s) configured to operate the source of pressurizing fluid to transfer fluid from the fluid reservoir to the lumen of said inflatable tubular member, via at least one of: push forces, and pull forces, applied thereon said actuating mean(s).

According to some embodiments, the inflatable tubular member is flexible and inflatable in response to fluid transfer from the reservoir into the lumen defined within the inflatable tubular member.

According to some embodiments, the forces are applied on the actuating means by a subject manually sliding along the penis in repeated alternate proximal and distal directions.

According to some embodiments, the actuating mean(s) includes a bar disposed within and extending from a distal end of the lumen of the inflatable tubular member.

According to some embodiments, the bar is in the form of a piston having a rod distally attached to a laterally protruding piston head.

According to some embodiments, the applying push and/or pull forces includes grasping the piston head and pushing and/or pulling the head through the penis.

According to some embodiments, the actuating mean(s) is being connected directly or via a pump activator to the source of pressurizing fluid.

According to some embodiments, the source of pressurizing fluid is a fluid pump.

According to some embodiments, the fluid pump is a diaphragm pump comprising a flexible diaphragm and configured for pumping fluid from the reservoir to the inflatable tubular member in response to reciprocate displacements of the diaphragm through transmission of the push and/or pull forces via the actuating means.

According to some embodiments, the diaphragm separates between a first chamber and a second chamber, and the pump further comprising at least one valve positioned within the first chamber and configured to close an inlet of the reservoir and to allow fluid flow there through when pull forces are applied on the diaphragm and to restrict fluid flow there through when push forces are applied on the diaphragm.

According to some embodiments, the reservoir is proximally integrated with the inflatable tubular member and the source of pressurizing fluid is contained within or is directly linked with the reservoir, thereby forming a one-piece penile prosthesis implant.

According to some embodiments, the bar is formed from a resilient material, such as but not limited to, silicone.

According to some embodiments, the prosthesis further comprises a pump activator for connecting the bar with the source of pressurizing fluid.

According to some embodiments, the pump activator is fluidly connected with the source of pressurizing fluid and the inflatable tubular member.

According to some embodiments, the inflatable tubular member is made of a flexible material.

According to some embodiments, the fluid is selected from silicone oil, saline, and water.

According to some embodiments, the prosthesis further comprises means to anchor at least a portion of the implant with a penis tissue.

According to some embodiments, the prosthesis further comprises at least one valve for controllably transferring fluid from one compartment of the implant to another compartment of the implant.

According to another aspect, the present invention provides a method for effecting a penis erection in a subject in need thereof, the method including providing a penile prosthesis configured to effect erection when push and/or pull forces are applied on the prosthesis by a subject through the penis; implanting the penile prosthesis within the penis of the subject; and applying push and/or pull forces on the penis to thereby effect an erection.

According to some embodiments, the forces are repeated forces.

According to some embodiments, the applying push and/or pull forces comprises manually sliding along said penis shaft in repeated alternate proximal and distal directions.

According to some embodiments, the penile prosthesis comprises: an inflatable tubular member defining a lumen; a source of pressurizing fluid; a fluid reservoir for holding fluid and in fluid communication with the source of pressurizing fluid and the inflatable tubular member; and actuating mean(s) configured to operate the source of pressurizing fluid to transfer fluid from the fluid reservoir to the lumen of the inflatable tubular member, via at least one of said push forces, and pull forces, applied thereon the actuating mean(s).

According to some embodiments, the actuating mean(s) includes a bar disposed within a lumen of the inflatable tubular member.

According to some embodiments, the bar is in the form of a piston having a rod distally attached to a laterally protruding piston head.

According to some embodiments, the push and/or pull forces includes grasping the head through the penis and pushing and/or pulling the penis.

According to some embodiments, the reservoir is integrally proximally connected with the inflatable tubular member to thereby form a monolithic implant, and the implanting includes positioning the reservoir to extend from a root of the corpora cavernosum of the penis and the inflatable tubular member to extend from a distal end of the corpora cavernosum.

According to some embodiments, the source of pressurizing fluid is a diaphragm pump configured for pumping fluid from the reservoir to the inflatable tubular member in response to the push and/or pull forces.

According to some embodiments, the source of pressurizing fluid is a corrugated pump configured for pumping fluid from the reservoir to the inflatable tubular member in response to the push and/or pull forces.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1B are schematic side cut views showing a penile prosthesis having an inflatable tubular member, a reservoir, a source of pressurizing fluid, and a bar operably linked with the source of pressurizing fluid, in a non-erect, flaccid state (FIG. 1A) and in an erect rigid state (FIG. 1B), in accordance with some embodiments of the invention;

FIGS. 2A-2B are schematic side cut views showing a penile prosthesis in which a source of pressurizing fluid is positioned proximally to an inflatable tubular member, in a non-erect, flaccid state (FIG. 2A) and in an erect rigid state (FIG. 2B), in accordance with some embodiments of the invention;

FIGS. 3 is a schematic cross section view showing a penile prosthesis of the invention, wherein the source of pressurizing fluid is a diaphragm pump, in accordance with some embodiments of the invention;

FIGS. 4A-4B show a cross section view of a penile prosthesis of the invention with an exemplary diaphragm pump (FIG. 4A), and a close view (FIG. 4B) showing the exemplary diaphragm pump, in accordance with some embodiments of the invention.

FIGS. 5A-5B show a schematic side view (FIG. 5A) and a cross sectional side view (FIG. 5B) of a penile implantable prosthesis having an inflatable tubular member, a reservoir, and a tubing system operable to transfer fluid from the reservoir to the inflatable tubular member in response to extortion forces applied by a subject, in accordance with some embodiments of the invention;

FIGS. 6A-6B show a schematic side view (FIG. 6A) and a cross sectional front view (FIG. 6B) of a tubing system of a penile prosthesis, in accordance with some embodiments of the invention;

FIGS. 7A-7B show an isometric view (FIG. 7A) of inflatable tubular member, and an isometric cross section view (FIG. 7B) of an inflatable tubular member surrounded by tubing system, in accordance with some embodiments of the invention;

FIGS. 8A-8B show a schematic side, partially exploded, view (FIG. 8A) and a cross sectional side, partially exploded, view (FIG. 8B) of a distal portion of the penile prosthesis of the invention having an inflatable tubular member, a tubing system and at least one valve, in accordance with some embodiments of the invention;

FIGS. 9A-9B show a schematic side, partially exploded, view (FIG. 9A), and cross sectional side, partially exploded, view (FIG. 9B) of a distal portion of a penile prosthesis of the invention that has an inflatable tubular member, a tubing system, at least one valve, and a manifold, in accordance with some embodiments of the invention;

FIG. 9C is an isometric view of a manifold, in accordance with some embodiments of the invention;

FIG. 10A is a schematic side, partially exploded, view of the penile prosthesis of the invention having an inflatable tubular member, a reservoir, a tubing system, at least one valve, a manifold, and a rod member disposed within the reservoir, in accordance with some embodiments of the invention;

FIGS. 10B-10C show a schematic side view (FIG. 10B) and cross sectional side view (FIG. 10C) of a rod member connected with a discharge fluid system, in accordance with some embodiments of the invention;

FIGS. 10D-10F show an isometric view (FIG. 10D) a front view (FIG. 10E), and a cross sectional side view (FIG. 10F) of a plug member surrounded by a disc member of discharge fluid system of a penile prosthesis, in accordance with some embodiments of the invention;

FIGS. 11A-11B show a schematic side view (FIG. 11A) of the penile prosthesis that includes a column member disposed within a tubular member and a cross sectional front view (FIG. 11B) of the tubular member surrounded by a tubing system, in accordance with some embodiments of the invention;

FIG. 12 is a simplified illustration of a penile prosthesis, in accordance with another non-limiting embodiment of the invention;

FIG. 13 is a simplified illustration of a penile prosthesis, in accordance with another non-limiting embodiment of the invention; and

FIG. 13A is a simplified illustration of a variation of the embodiment of FIG. 12; FIG. 13B is a simplified illustration of fluid paths of a high pressure lumen and a low pressure lumen associated with the reservoir of FIG. 13A;

FIGS. 13C-13G are simplified illustrations of different kinds of proximal end caps which may be used as anchoring members for the prosthesis of FIG. 13A.

### DETAILED DESCRIPTION

The present invention, in some embodiments thereof, relates to inflatable penile prostheses, which operate to become erect and/or flaccid in a manner that mimics natural intercourse or male's masturbation. The present invention further relates to methods for using the penile prostheses of the invention for treating erectile dysfunction.

Erectile dysfunction (ED) or impotence is characterized by the inability to develop or maintain an erection of the penis during sexual activity. Typically, ED or impotence is treated with medications or penile prosthesis implantation within the penis.

Nevertheless, not all patients respond well to medications and the known penile prostheses are still associated with substantial drawbacks, such as, discomfort to the patient and/or inefficiency in achieving erection.

The present invention provides a simple, safe, reliable, easy to operate and comparatively inconspicuous implantable penile prosthesis, whereby the impotent male can achieve a penile erection that simulates natural erection process (i.e., the penile prosthesis causes the penis to become longer, thicker, harder and stiffer in response to manual push and/or pull forces of the subject along a longitudinal axis of the penis, those forces may be applied by sexual intercourse and/or masturbation). That is to say, the penile prosthesis of the invention achieves erection without requiring manual squeezing of a pump positioned within the scrotum. The present invention further provides a penile prosthesis, which may be a one-piece implant and that may be readily implanted into the corpus cavernosum of a male subject.

According to some embodiments, the penile prosthesis of the invention includes an inflatable tubular member and a fluid reservoir. According to some embodiments, the penile prosthesis of the invention further includes a source of pressurizing fluid (e.g., a fluid pump such as a diaphragm pump). According to some embodiments, the penile prosthesis of the invention transforms from a flaccid state to an erect state in response to fluid flow from a reservoir to the inflatable tubular member. According to some embodiments, the penile prosthesis of the invention returns to a flaccid state in response to fluid flowing back to the reservoir.

According to some embodiments, the penile prosthesis of the invention further includes actuating mean(s) configured to operate the source of pressurizing fluid. As used herein the actuating mean(s) refer to any compartment, feature, element or assembly that is operable or contributes to fluid transfer from the reservoir to the inflatable tubular member in response to push and/or pull forces. According to some embodiments, the actuating mean(s) include a bar operably linked to the source of pressurizing fluid. According to some embodiments, the bar is a piston. According to some embodiments, the piston includes a rod attached to a piston head. According to some embodiments, the pull and/or push forces activate the source of pressurizing fluid to suction fluid from the reservoir. According to some embodiments, the actuating mean(s) include a bar operably linked to the source of pressurizing fluid and the forces are pull forces. The term “bar” encompasses any slender element which may be employed to produce a pumping effect, such as but not limited to, a bar, rod, tube and the like, of any cross-sectional shape, and the terms bar and rod are used interchangeably.

According to some embodiments, an erection state of the prosthesis is induced by push and/or pull forces applied by the patient on the prosthesis. According to some embodiments, pushing or pulling the penis (e.g., in a sideward or upward direction) is mediated by a subject grasping, through the penis, a bar located within the penile implant, and pushing/pulling the bar.

According to some embodiments, the prosthesis is configured for implantation within the corpus cavernosum, wherein the fluid reservoir is implantable to extend from the proximal end of the corpus cavernosum (e.g., the root of the corpus cavernosum) and the inflatable member extends therefrom. Throughout the description, it is understood that the implant may be implanted in both the left and right corpus cavernosa, or just one of them, if the situation warrants such an implantation.

According to some embodiments, at least a portion of the penile prosthesis of the invention is coated with a hydrophilic material layer to allow water absorption from the surrounding body fluids and tissue.

According to some embodiments, at least a portion of the penile prosthesis of the invention is coated with antibiotics to thereby prevent possible infections following implantation.

According to some embodiments, the prosthesis is adjustable to various penis sizes. The dimensions of the penile prosthesis are not limited to any particular value.

According to some embodiments, the inflatable tubular member of the penile prosthesis of the invention in an erect state has a pressure that ranges between about 1000 mm Hg and about 1300 mm Hg. The invention is not limited to these values.

According to some embodiments, the prosthesis is resistant to wear and/or tear.

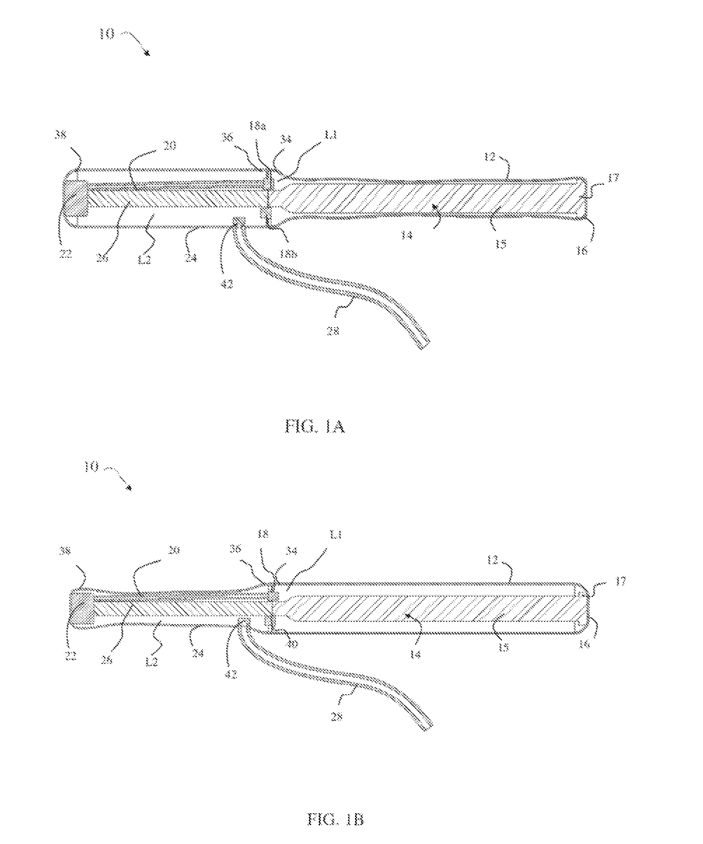

Reference is now made to FIGS. 1A-1B which show a cross-sectional side view of an exemplary penile prosthesis 10, in a non-erect, flaccid state (FIG. 1A) and in an erect rigid state (FIG. 1B), in accordance with some embodiments of the invention. It is to be understood that the typical application would include two penile prostheses 10 each of which suitable for implantation within the corpus cavernosum.

Penile prosthesis 10 includes an inflatable tubular member 12 extending from a proximal end 34 and a distal end 16 and defining a lumen L1. Penile prosthesis 10 further includes a source of fluid 22 (such as pressurizing fluid 22) and a reservoir 24. Reservoir 24 extends between a proximal end 38 and a distal end 36 and defines a lumen L2. Inflatable tubular member 12 houses actuating mean(s), such as bar 14, configured to operate the source of pressurizing fluid to transfer fluid from lumen L2 to lumen L1 of inflatable tubular member 12. Optionally, the length ratio between reservoir 24 and the inflatable tubular member 12 is between about 1:4 and about 1:1. According to some embodiments, the length ratio between reservoir 24 and the inflatable tubular member 12 is about 1:2, or about 1:1. Generally, penile prosthesis 10 may be implanted entirely within the corpus cavernosum of a patient suffering from impotence, wherein reservoir 24 is implanted to extend from the proximal end of the corpus cavernosum and inflatable tubular member 12 is implanted to extend from the distal end of the corpus cavernosum.

Bar 14 is operably connected or linked with source of pressurizing fluid 22. Bar 14 may be in the form of a piston and may include a rod member 15 attached to a distal laterally protruding piston head 17.

Referring to FIG. 1B, the penile prosthesis 10 is illustrated in an erect state, wherein inflatable tubular member 12 displaced a distance away from bar 14. Penile prosthesis 10 is operable to induce a penile erection via bar 14, which during sexual activity, applies push and/or pull forces at source of pressurizing fluid 22. Optionally the push and/or pull forces are applied through piston head 17 that can be stably grasped and pulled and/or pushed by a subject. The piston, piston rod or piston head are just some examples of a movable actuating element which is operative to transfer the fluid from the fluid reservoir to the inflatable tubular member upon axial movement of the movable actuating element along the longitudinal axis of the penile implant. In response to said forces, pressurizing fluid 22 becomes actuated and forces fluid transfer from lumen L2 of reservoir 24 to lumen L1 of inflatable tubular member 12.

As used herein the term “operably connected or linked” relates to the actuating mean(s), such as bar 14, being capable of performing work on or capable of effecting the actuation of source of pressurizing fluid 22 to pump and transfer fluid from reservoir 24 to inflatable member 12. Thus, bar 14 is configured such that when push and/or pull forces are applied thereon, source of pressurizing fluid 22 is operated and fluid is transferred to inflatable tubular member 12.

As used herein, the term “push forces” relates to those forces, applied by a subject to push bar 14 proximally towards the body.

As used herein, the term “pull forces” relates to those forces, applied by a subject to pull bar 14 distally and away from the body.

As used herein the term “proximal” means that the referenced part is situated next to or near the point of attachment or origin or a central point: as located toward a center of the human body.

As used herein the term “distal” means that the referenced part is situated away from the point of attachment or origin or the central point: as located away from the center of the human body. A distal end is the furthest endmost location of a distal portion of a thing being described, whereas a proximal end is the nearest endmost location of a proximal portion of the thing being described.

According to some embodiment, inflation of inflatable tubular member 12 requires sequential or reciprocating or repeated push/pull (or back and forth) forces on bar 14 (also referred to as rod or piston 14). The push and pull forces effect pumping of source of pressurizing to pump fluid.

According to some embodiment, complete inflation of inflatable tubular member 12 achieves a diameter of about 15 mm of inflatable tubular member 12.

Referring to FIG. 1A, the penile prosthesis 10 is illustrated in a flaccid state. The inflatable tubular member 12 is relaxed and may be wrinkled or crumpled. At this configuration, the inflatable tubular member 12 might even contact bar 14. Penile prosthesis 10 may be deflated to shift to a flaccid state by manually draining inflatable tubular member 12 (e.g. by a subject squeezing) proximally, towards the reservoir and/or by bending the penis in a sharp angle (e.g. downward) to thereby force fluid transfer from inflatable member 12 and back to the reservoir 24.

Reservoir 24 defines a lumen L2 and is capable of holding fluid. Optionally, reservoir 24 includes a stiff rod (not shown) to facilitate stability and avoid collapse of reservoir 24 when the implant is in a flaccid state. According to some embodiments, reservoir 24 is proximally integrated with inflatable tubular member 12 to thereby form a monolithic or a one-piece implant. Source of pressurizing liquid 22 may be included in either reservoir 24 or in inflatable tubular member 12 or may be located in between reservoir 24 and inflatable tubular member 12.

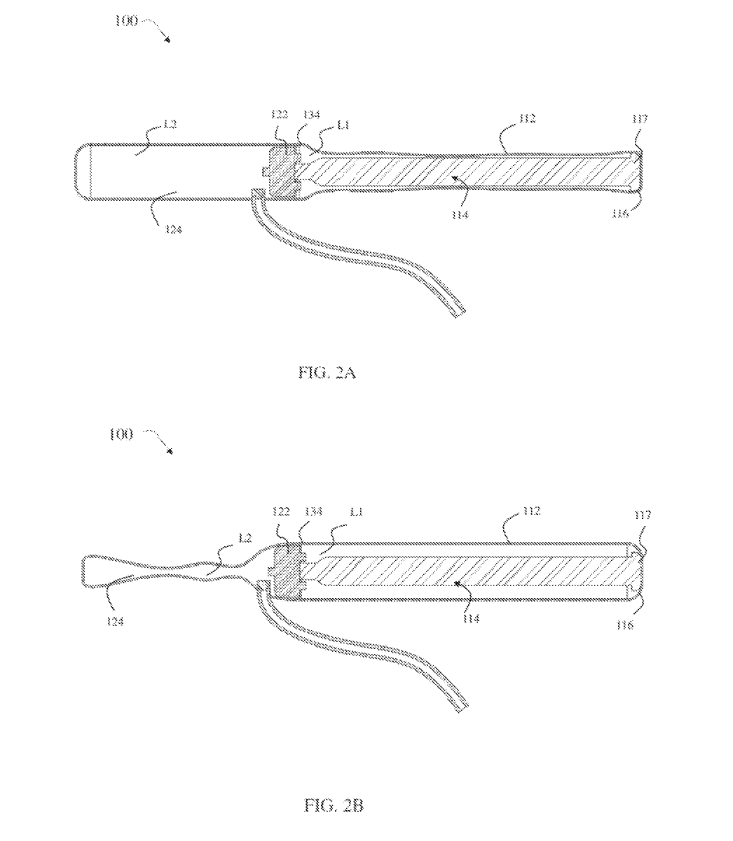

According to some embodiments, inflatable tubular member and bar 14 are arranged in a coaxial manner with inflatable tubular member 12 disposed around bar 14. Bar 14 may extend between inflatable tubular member distal end 16 and inflatable tubular member proximal end 34. According to some embodiments, inflatable tubular member 12, defines a lumen L1 and bar 14 is positioned within lumen L1. Bar 14 is in communication with a source of pressurizing fluid 22. Bar 14 may be directly connected with source of pressurizing fluid 22 (as shown in FIGS. 2A-2B). Alternatively, and as shown in FIGS. 1A-1B, bar 14 may be indirectly connected with source of pressurizing fluid 22. In accordance with this embodiment, bar 14 is positioned within tubular member 12 and is linked with source of pressurizing fluid 22 via a pump activator 26.

Bar 14 may be fabricated from a resilient material to allow bending and flexibility of a penis. Suitable resilient materials include, but are not limited to silicone, and thermoplastic elastomers. Further optionally, bar 14 is segmented to include a plurality of separate segments linked together, to facilitate flexibility and bending.

Inflatable tubular member 12 may be substantially cylindrical in shape. Inflatable tubular member 12 may be made from an elastic, flexible material including, but not limited to, an elastomer (e.g. polyurethane or silicone rubber). Inflatable tubular member 12 may be coated with a hydrophilic material to allow expansion within the corpus cavernosum tissue following implantation. Either or both inflatable tubular member 12 and reservoir 24 may be coated with antibiotics to prevent possible infections following implantation.

According to some embodiments, the inflatable tubular member 12 has a wall with a thickness that ranges between about 0.2-0.5 mm, 0.5 mm and about 3 mm, between about 0.2 mm and about 2.5 mm, between about 0.2 mm and about 2 mm, between about 0.2 mm and about 1.5 mm, or between about 0.2 mm and about 1 mm. Each possibility represents a separate embodiment of the invention. In all embodiments, the prosthesis may have a hydrophilic coating. This particular wall thickness may provide an inflatable tubular member 12 that may be expanded to facilitate erection, yet prevents deformation associated with over expansion of the inflatable tubular member. Further, this particular wall thickness is also configured to limit the fluid volume capacity needed for effecting erection, thus allowing minimalizing the size of the reservoir 24.

Inflatable tubular member 12 may have a length that ranges between about 8 cm and about 21 cm, between about 8 cm and about 16 cm, or between about 8 cm and about 12 cm. Each possibility represents a separate embodiment of the invention.

Inflatable tubular member 12 becomes inflated via activation of source of pressurizing fluid 22 and fluid transfer from the reservoir 24 to the inflatable tubular member 12. At an erect state, inflatable tubular member 12 may have an outer diameter that ranges between about 10 mm and about 17 mm, or between about 12 mm and about 15 mm. Each possibility represents a separate embodiment of the invention. Inflatable tubular member 12 becomes deflated following fluid transfer from the inflatable tubular member 12 back to the reservoir 24. At the flaccid state, inflatable tubular member 12 may have a diameter that ranges between about 8 mm and about 13 mm. The dimensions of the prosthesis are not limited to any particular value.

According to some embodiments, the reservoir 24 may, but not necessarily, be provided as a cylindrical reservoir formed from an elastic, flexible polymer. Alternatively, reservoir 24 may be substantially solid. Reservoir 24 is sized and shaped to contain a liquid such as saline.

Reservoir 24 is sized and shaped to be minimal in size, holding a minimal volume of liquid as compared to fluid reservoirs of known penile prostheses, yet affording erection which is long, thick, and stiff. Those particularly small dimensions of the reservoir 24 afford increased comfort to the patient and provide an implant, which may be readily, optionally entirely, implanted within the corpus cavernosum. The reservoir 24 is sized to hold a volume of liquid of between about 3 ml and about 20 ml. According to some embodiments, the reservoir 24 is sized to contain a volume of about 20 ml, about 15 ml, about 10 ml or about 5 ml. Each possibility represents a separate embodiment of the invention. According to some embodiments, reservoir 24 has a length of no more than about 7 cm.

According to some embodiments, reservoir 24 has an outer diameter of between about 10 mm and about 21 mm, or between about 14 mm and about 18 mm. According to some embodiments, reservoir 24 has a maximal outer diameter of no more than about 18 mm.

According to some embodiments, the source of pressurizing fluid 22 is a fluid pump, such as a diaphragm pump. The source of pressurizing fluid 22 may be positioned within reservoir 24 and may be located at various positions within same. According to some embodiments, source of pressurizing fluid 22 is positioned at the proximal end 38 or distal end 36 of reservoir 24. The pump 22 is operable to transfer liquid from the reservoir 24 into the inflatable member 12 to thereby fill lumen L1 defined between inflatable member 12 and bar 14. The pump 22 is operable in response to push and/or pull forces applied through bar 14 and particularly by a patient while grasping head 17 through the penis.

The source of pressurizing fluid 22 may be connected to inflatable member 12 via at least one reservoir tube 20. Tube 20 is configured to transfer fluid from the reservoir 24 to the inflatable member 12 and may optionally be configured to transfer fluid from the inflatable member 12 back into reservoir 24. Optionally, the penile prosthesis includes at least one valve 18 for directing or controlling fluid flow by opening, closing, or partially obstructing fluid passage there through.

According to some embodiments, penile prosthesis 10 includes two valve 18a and valve 18b, each of which may be located at different positions, optionally at two opposite sides within reservoir 24. Valves 18a an 18b may be configured to allow fluid flow to only one direction. Further optionally, at least one of valves 18a and 18b is a one-way valve. Valves 18a and 18b may allow liquid transfer between inflatable tubular member 12 and reservoir 24. Valve 18a may allow liquid transfer from reservoir 24 to inflatable tubular member 12 via tube 20 and may prevent liquid flowing backward to reservoir 24. Valve 18b may allow liquid transfer from inflatable tubular member 12 to reservoir 24 directly or via a tube (such as tube 20, not shown) and may prevent liquid flowing backward to inflatable tubular member 12.

Due to various penis lengths between patients, the penile prosthesis 10 may further include a tip (not shown) configured to extend the penile implant in the distal or proximal direction, for an improved fit. The tip may be a rear tip or a frontal tip. Optionally, the tip is a tapered tip.

Penile prosthesis 10 may further include a filling tube 28 and a plug 42 for allowing filling reservoir 24 through the course of implantation of the penile prosthesis within the scrotum cavernosa. Following completion of filling reservoir 24 with the fluid, filling tube 28 is detached and plug 42 is allowed to be closed, thus sealing filling tube 28 opening.

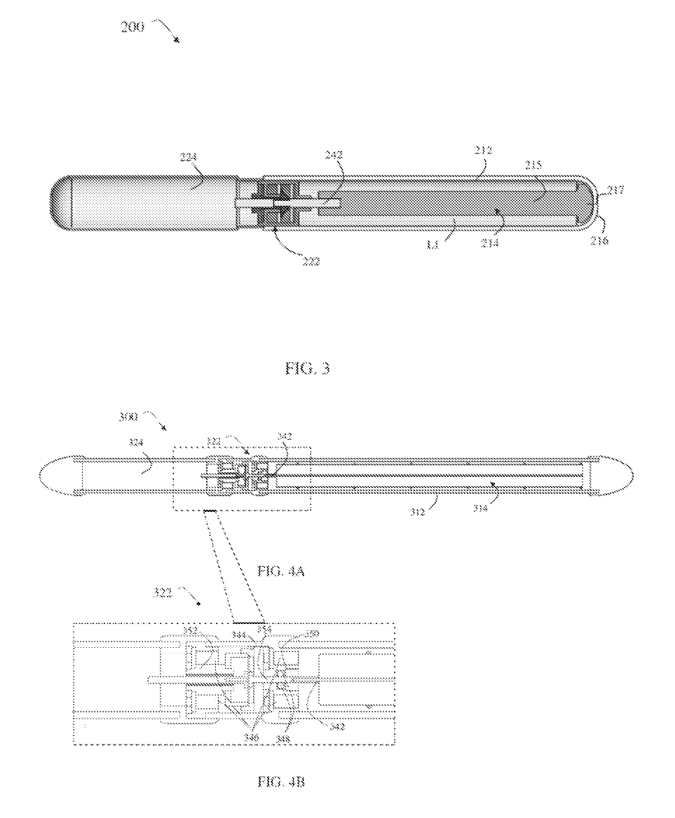

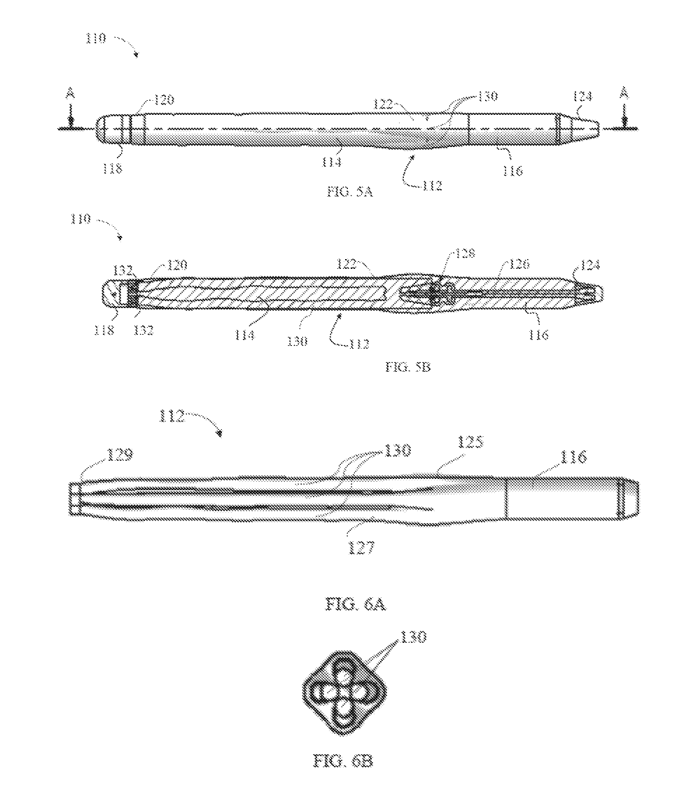

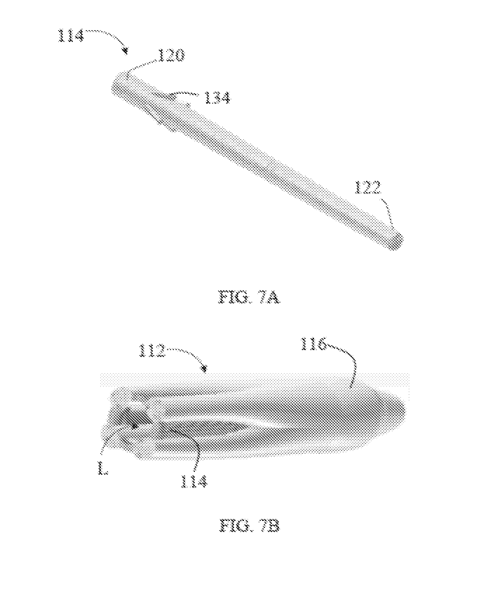

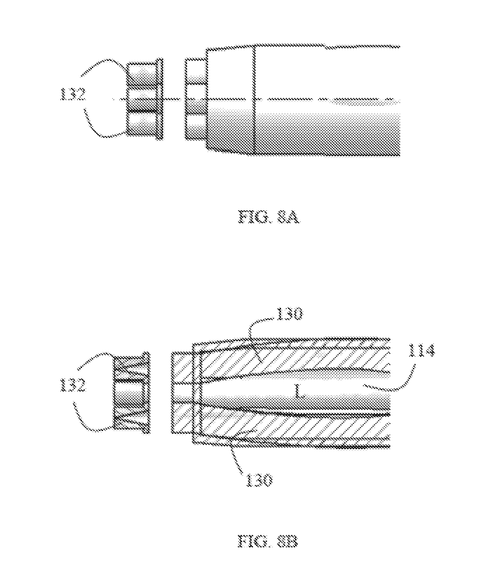

Reference is now made to FIGS. 2A-2B which illustrate penile prosthesis implant 100 according to some embodiments of the invention. Penile prosthesis implant 100 is similar to penile prosthesis implant 10, except for source of pressurizing liquid 122 that is located adjacent the proximal end 134 of inflatable tubular member 112, or between reservoir 124 and inflatable tubular member 112. Similarly to penile prosthesis 10, penile prosthesis 100 includes a bar 114 disposed within lumen L1 defined by inflatable tubular member 112. Bar 114 may extend between inflatable tubular member distal end and proximal end, 116 and 134, respectively. In the Figs bar 114 is shown to connect source of pressuring liquid 122 directly, but bar 114 may also be connected with source of pressurizing fluid 122 indirectly (shown in FIGS. 3-4).

Referencing to FIG. 2B, penile prosthesis 100 is shown in an erect state. Similar to penile prosthesis 10, penile prosthesis 100 may be operated to induce erection by performing at least one of push and/or pull of forces on bar 114, optionally through piston head 117. By pushing/pulling head 117, source of pressurizing liquid 122 is actuated. Further, upon activating source of pressurizing liquid 122, liquid is forced to transfer from reservoir 124 to inflatable tubular member 112 to thereby induce erection.

Referencing to FIG. 2A, penile prosthesis 100 is shown in a flaccid state. Further similar to penile prosthesis 10, deflation may optionally be achieved by squeezing the penis and/or bending the penis to thereby force liquid transfer from inflatable tubular member 112 into reservoir 124.

Reference is now made to FIG. 3 showing an exemplary penile prosthesis 200, wherein the source of pressurizing fluid is a diaphragm pump, in accordance with some embodiments of the invention.

Similarly, to penile prostheses 10 and 100, penile prosthesis 200 has a reservoir 224, an inflatable tubular member 212 defining a lumen L1 and housing bar 214. Bar 214 may be in the form of a piston and may have rod member 215 attached to a piston head 217 located at the distal end 216 of inflatable tubular member 212 and configured to allow stable grasping through the penis by a subject. Penile prosthesis 200 includes a diaphragm pump 222 that may optionally be positioned between inflatable tubular member 212 and reservoir 224. Diaphragm pump 222 may be connected with bar 214 via pump activator 242. Pump activator 242 may optionally be tubular, and may define a lumen. Pump activator 242 may optionally include an inner valve.

Advantageously, diaphragm pump 222 may effect fluid pumping and transferring towards only one direction only (i.e., from reservoir 224 to inflatable tubular member 212). An exemplary diaphragm pump, elements thereof and operation mode will be explained herein below with reference to FIG. 4.

Reference is now made to FIGS. 4A-4B showing an exemplary penile prosthesis 300, wherein the source of pressurizing fluid is a diaphragm pump, in accordance with some embodiments of the invention

Penile prosthesis 300 is similar to penile prosthesis 200 and having an exemplary diaphragm pump 322. Diaphragm pump 322 includes a diaphragm 344 optionally attached to pump piston 354. Diaphragm 344 is flexible and may be formed from any suitable elastomeric material that is not deleteriously affected by the fluids intended to be used with the pump action. Diaphragm 344 separates between first chamber 352 and second chamber 350. Diaphragm pump 322 further includes at least one valve 346, (optionally a duckbill valve). Valves 346 are one-way valves that allow fluid to flow only in one direction (i.e., from reservoir 324 to first chamber 352, and/or second chamber 350 and/or inflatable tubular member 312). Optionally, diaphragm pump 322 includes at least one ring 348 configured to seal chamber 350. Diaphragm pump 322 is operated in response to sequential push-pull forces applied by a subject on bar 314. The push and pull forces facilitate proximal and distal, optionally with an upward or downward direction, movements of bar 314, respectively. The forces are facilitated either directly or via pump activator 342 or via pump piston 354 on diaphragm 344. Pump activator 342 may be attached to diaphragm 344 directly, or indirectly via a piston 354.

In operation of the pump 322, fluid is drawn into first chamber 352 in response to diaphragm 344 being pulled distally and enlargement of first chamber 352. Such action establishes a vacuum condition within first chamber 352 that causes at least one of valves 346 located within the first chamber 352 to open and allow fluid flow from reservoir 324 to first chamber 352. When push forces are applied on diaphragm 344 to be pushed proximally, fluid within first chamber 352 is forced to flow and fill second chamber 350 and from there through valves 346 located within second chamber 350 to inflate inflatable tubular member 312.

The push-pull (or back and forth movements) of the diaphragm 344 in opposite directions thus establish a one way flow of fluid through first chamber 352, and second chamber 350 and valves 346.

According to some embodiments, pump activator 342 is hollow and may include an internal valve.

The present invention further provides methods of inducing an erection in a subject in need thereof and methods of treating erectile dysfunction or impotence. The method presented hereinbelow includes a step of implanting an inflatable penile prosthesis, such as inflatable penile prosthesis 10, 100, 200, and 300 of the invention. Nevertheless, the method presented hereinbelow is described with reference to penile prosthesis 10, but is applicable to any of the above mentioned prostheses.

Implanting the penile prosthesis of the invention includes the surgeon forming incisions to access and expose the corpora cavernosum of the patient. The corpora cavernosum is then dilated (e.g., by introducing a dilator into the spongy tissue of the corpora) to create a space for the penile prosthesis. The surgeon then measures a length of the dilated corpora cavernosum to allow adjustment of the length of the penile prosthesis by adjusting the size of a tip attached to the implant. After dilation of the corpus cavernosum, a penile prosthesis is inserted into the prepared corpora cavernosum with the reservoir portion placed adjacent to, or at, the penis crust or root of corpora cavernosum. The reservoir may optionally then be filled with liquid via filling tube such as filling tube 28. Thereafter, the filling tube is detached and a plug, such as plugs 42 is allowed to seal the opening of the filling tube to avoid liquid leakage.

During use of the implant, a subject, when applying push and/or pull forces on bar 14 actuates source of pressurizing fluid 22 which in response operates to force fluid flow from reservoir 24 into the inflatable tubular member to inflate the penis from a flaccid state to an erect state.

The valve 18 is configured to allow the fluid to flow from the reservoir 24 into the inflatable tubular member 12 and impede reverse fluid flow. In accordance with this embodiment, valve 18 may be a one-way valve or a check valve.

When referring to penile prostheses 200 and 300 that includes a diaphragm pump, (shown in FIGS. 3-4), an erect state may be achieved following sequential push-pull forces applied on bar 214 or 314, respectively, by the subject. Those forces operate diaphragm pump 222 or 322 to pump fluid from reservoir 224 or 324 to inflatable tubular member 212 and 312.

For returning to a flaccid state, the patient may squeeze or bend the penis downward to thereby allow the return flow of the fluid.

Reference is now made to FIGS. 5A-5B which show a schematic side view (FIG. 5A) and a schematic cross sectional side view (FIG. 5B) of an exemplary penile prosthesis 110, in accordance with some embodiments of the invention. It is to be understood that the typical application would include two implantable penile prostheses 110 each of which suitable for implantation within the corpus caverno sum. According to some embodiments, the prosthesis is a two-piece implant, wherein the reservoir is fluidly connected with the tubular member and is implantable within the abdomen and the tubular member is implantable within the penis of a subject.

According to some embodiments, the prosthesis is a one-piece implant, wherein the reservoir is integrated with the tubing system and/or the tubular member and the prosthesis is implanted within the corpora cavernosum, such that the fluid reservoir is implanted to extend from the proximal end of the corpus cavernosum (e.g., the root of the corpus cavernosum) and the inflatable member is in continuation thereof.

Penile prosthesis 110 includes an inflatable tubular member 114 extending from a proximal end 122 to a distal end 120 and defining a lumen (shown for example in FIG. 7B). Penile prosthesis 110 further includes a reservoir 116 for holding and storing fluid. Penile prosthesis 110 further includes a tubing system 112. According to some embodiments, tubing system 112 is disposed on a periphery or on a circumference of the inflatable tubular member 114. According to some embodiments, tubing system 112 is disposed on a circumference surface of the inflatable tubular member 114. Tubing system 112 may extend from a proximal end 122 to a distal end 120 of the inflatable tubular member 114. Tubing system 112 may be entirely external to tubular member 114. Alternatively, or optionally tubing system 112 extends from proximal end 122 of tubular member 114 and gradually integrates with tubular member 114 toward distal end 120 of tubular member 114. Tubing system 112 fluidly connects between reservoir 116 and inflatable tubular member 114. According to some embodiments, reservoir 116 is proximally connected with tubular member 114. According to some embodiments, reservoir 116 directly connects tubular member 114. Optionally, reservoir 116 is integrally connected with tubular member 114.

Penile prosthesis 110 may further include a proximal tip 124 optionally housing rod member 126 that provides stability and anchoring to implant 110 within the corpora cavernosum when the implant is in a flaccid state.

Further optionally, penile prosthesis 110 includes a distal tip 118. Optionally, penile prosthesis 110 may further include a rear tip provided to extend the penile length, optionally, in the proximal direction, for an improved fit (not shown).

According to some embodiments, tubing system 112 includes at least one tube 130 located at the periphery of the tubular member 114 and extending along a longitudinal axis A of penile prosthesis 110. Optionally, tubing system 112 includes at least two, at least three, at least four, at least five, or at least six tubes 130. Each possibility represents a separate embodiment of the invention. Further optionally, tubing system includes at least one valve 132 disposed at a distal end of tube 130 and configured to allow fluid flow to one direction (i.e., towards inflatable tubular member 114). According to some embodiments, valve 132 is a one-way valve (e.g. a duckbill valve). Valve 132 may prevent liquid flowing backward to tubing system 112 and/or reservoir 116.

As in all embodiments of the invention, the inflatable tubular member may be made from an elastic, flexible material including, but not limited to, an elastomer (e.g. polyurethane or silicone rubber), and may be coated with a hydrophilic material to allow expansion within the corpus cavernosum tissue following implantation. The inflatable tubular member and/or reservoir may be coated with antibiotics to prevent possible infections following implantation.

Due to various penis lengths between patients, the penile prosthesis 110 may further include a tip (not shown) configured to extend the penile implant in the distal or proximal direction, for an improved fit. The tip may be a rear tip or a frontal tip. Optionally, the tip is a tapered tip.

According to some embodiments, implant 110 includes draining system 128 for draining fluid from tubular member 114 back into reservoir 116. Draining system 128 allows deflation of tubular member 114 such that the implant transforms to a flaccid state. Draining system 128 may be operated by manually draining inflatable tubular member 114 (e.g. by a subject squeezing) in a proximal direction, towards the reservoir 116 and/or by bending the penis in a sharp angle (e.g. downward) to thereby force fluid transfer out of the inflatable member 114 and back to the reservoir 116.

Reference is now made to FIGS. 6A-6B showing a schematic side view (FIG. 6A) and a front cross sectional view (FIG. 6B) of tubing system 112.

Referring to FIG. 6A, tubing system 112 includes at least one longitudinal tube 130 having a tubular wall and defining a lumen for flowing fluid. Tube 130 extends from a tube distal end 129 and a tube proximal end 127. According to some embodiments, tubing system 112 includes a plurality of tubes 130. According to some embodiments, tubing system 112 includes three or more, four or more, five or more or six or more tubes 130. Each possibility represents a separate embodiment of the invention. According to some embodiments, tubing system 112 is fluidly connected with reservoir 116. According to some embodiments, tubing system 112 is connected with a distal end 125 of reservoir 116.

According to some embodiments, tubing system 112 and reservoir 116 are integrally formed from same material, thereby forming a one-piece component. According to some embodiment, tubing system 112 includes tubes 130 each connected to distal end 120 of inflatable tubular member 114. According to some embodiment, reservoir 116 is a separate unit, optionally configured for implantation within the scrotum or abdomen of the subject.

Referring to FIG. 6B, front cross section of tubing system 112 is shown, wherein tubing system includes four tubes 130 that may be equally distanced from each other.

Reference is now made to FIGS. 7A-7B which show an isometric view of inflatable tubular member 114 (FIG. 7A) and a cross sectional isometric view of penile implant 110 of the invention in which tubing system 112 surrounds tubular member 114 (FIG. 7B).

Referring to FIG. 7A, tubular member 114 extends from a proximal end 122 to a distal end 120. Tubular member 114 is inflatable and flexible such that when filled with fluid, the cross sectional diameter of tubular member 114 is increased. According to some embodiments, without limitation, the overall fluid capacity of tubular member 114 is about 15 ml, about 20 ml, about 25 ml or about 30 ml or about 35 ml. Each possibility represents a separate embodiment of the invention. According to some embodiments, tubular member 114 is made from a polymer (e.g., polyurethane or silicone). According to some embodiments, tubular member 114 comprises an outer surface being rough, optionally having a wave-like texture allowing an increased surface area. According to some embodiments, tubular member 114 includes at least one sleeve 134, defining a lumen and configured for connecting tubes 130 of tubing system 112 with an inner lumen of the tubular member 114.

Referring to FIG. 7B, a cross section of penile implant 110 of the invention in which tubing system 112 surrounds tubular member 114. Tubular member 114 defines a lumen LL. As described above, tubing system 112 includes at least one tube 130 disposed on the circumference of tubular member 114.

Reference is now made to FIGS. 8A-8B showing a side view (FIG. 8A) and a cross section (FIG. 8B) of penile prosthesis 110, and showing tubing system 112, valve 132, reservoir 116 and tubular member 114.

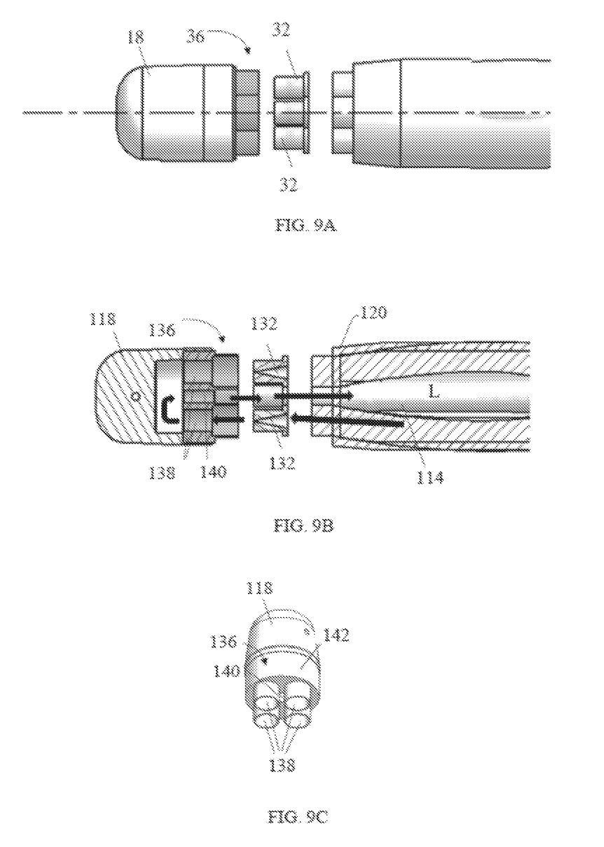

Reference is now made to FIGS. 9A-9C which show penile prosthesis 110 in accordance with some embodiments of the invention. Prosthesis 110 optionally includes manifold 136 for directing fluid flow from tubing system 112 and/or at least one tube 130 to the lumen of tubular member 114. According to some embodiments, manifold 136 is disposed in a distal end 120 of the tubular member 114. According to some embodiments, the manifold 136 includes at least one channel 138 and a manifold body 142. Channel 138 is in fluid communication and/or is configured for directing fluid from tube 130 and/or tubing system 112 to flow towards tubular member 114. According to some embodiments, manifold 136 includes a plurality of channels 138. According to some embodiments, manifold 136 includes at least three channels, at least four, at least five, or at least six channels. Each possibility represents a separate embodiment of the invention. According to some embodiments, manifold 136 includes a collector channel 140 that collects fluid flowing from tubing system 112 through channel(s) 138, and directs the fluid to flow and inflate tubular member 114. According to some embodiments, channel(s) 138 may be circumferentially disposed within manifold body 142 and collector channel 140 may be disposed at the center of manifold body 142.

Referring to FIG. 9C, manifold 136 is shown as a separate unit that includes four circumferential channels 138 and one central collector channel 140. Manifold 136 may be integrated or connected with tip 118.

Referring to FIG. 9B fluid flow direction is shown following a subject act of squeezing the penis from the proximal to the distal direction. Fluid flow is induced to exit reservoir 116, enter and flow through tubing system 112, optionally enter and flow through at least one of the channels 138 of manifold 136, optionally through at least one valve 132, and then directed to enter inflatable tubular member 114, optionally, through central channel 140 of manifold 136.

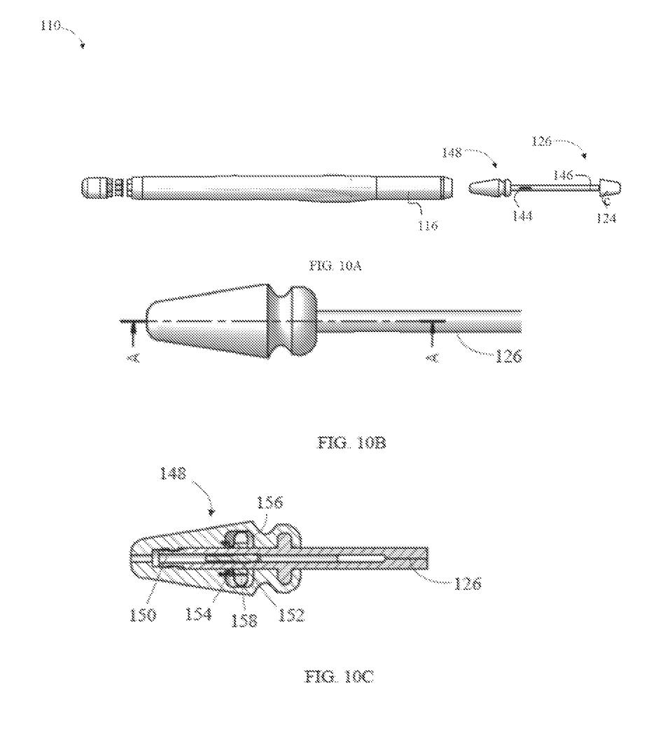

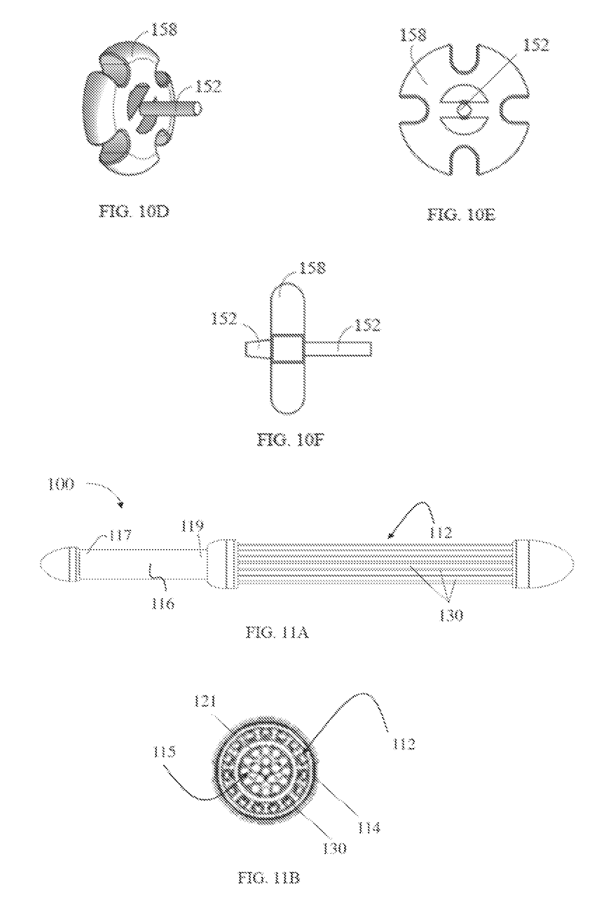

Reference is now made to FIGS. 10A-10F showing penile prosthesis 110 of the invention that may optionally include rod member 126 and fluid discharge system 148.

Referring to FIG. 10A, rod member 126 may extend from a rod distal end 144 and a rod proximal end 146, optionally positioned within reservoir 116 and configured to stabilize the implant and its position within the corpus cavernosum when reservoir 116 is devoid of fluid or emptied. Proximal end 146 of rod member 126 may be attached to proximal tip 124. Distal end 144 of rod member 146 may be attached and/or included within discharge system 148. Rod member 126 may define bore 150 (shown in FIG. 10C) extending along a longitudinal axis A of rod 126. Bore 150 allows fluid communication between tubular member 114 and reservoir 116. Bore 150 thus allows fluid flow there through, optionally in one direction, towards reservoir 116.

Referring to FIG. 10B, a side view is shown of rod member 126 that is directly attached to discharge system 148.

Referring to FIG. 10C, a cross section is shown of rod member 126 that is directly attached to discharge system 148. Fluid discharge system 148 may include distal end 144 of rod member 126. Discharge system 148 may further include plug 152 and spring 154 disposed within chamber 156. Plug 152 may be integrally formed or connected with disc 158.

A flaccid state of the penile implant 110 of the invention is induced following a subject squeezing and/or sliding and/or milking along a longitudinal axis of the implant from a distal to the proximal direction and/or bending the penis. Fluid discharge system 148 is actuated when fluid pressure within bore 150 effects movement of plug 152 that in turn releases spring 154, allowing fluid flow towards reservoir 116.

Referring to FIGS. 10D-10F, plug 152 and disc 158 of discharge system 148 are shown. Plug 152 and disc 158 may be connected, optionally integrally structured, optionally from a single piece of material.

Reference is now made to FIGS. 11A-11B which show penile prosthesis 100 in accordance with some embodiment of the invention. Prosthesis 100 includes a reservoir 116, extending from a reservoir proximal end 117 to a reservoir distal end 119. prosthesis 100 further includes a tubing system 112 including plurality of tubes 130.

Referring to FIG. 11B, tubular member 114 may house a column 115 being composed from at least one malleable wire 121. Optionally at least one wire(s) 121(s) is segmented to include a plurality of separate segments linked together, to facilitate flexibility and bending.

As in all embodiments, erection is caused by axial movements (e.g., of the user's hand) on the outer contour of the penile prosthesis to pump fluid to fill the inflatable tubular member, thus inducing an erection. The valve 132 is configured to allow the fluid to flow from the reservoir 116 into the inflatable tubular member 114 and impede reverse fluid flow. In accordance with this embodiment, valve 132 may be a one-way valve or a check valve.

For returning to a flaccid state, the patient may squeeze or bend the penis downward to thereby allow the return flow of the fluid towards reservoir 116.

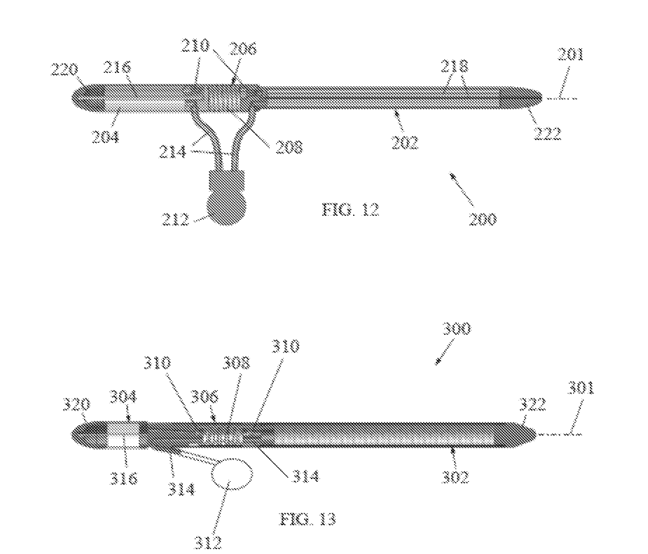

Reference is now made to FIG. 12, which illustrates a penile prosthesis 200, in accordance with a non-limiting embodiment of the invention.

Penile prosthesis 200 has a longitudinal axis 201 and includes an inflatable tubular member 202 which is more rigid when inflated than when deflated. Prosthesis 200 has a fluid reservoir 204 for holding fluid (such as, but not limited to, saline, air, water or other suitable liquids or gas). Fluid reservoir 204 is in fluid communication with inflatable tubular member 202.

A pump 206 includes a movable actuating element 208 operative to transfer the fluid from fluid reservoir 204 to inflate inflatable tubular member 202 upon axial movement of the movable actuating element 208 along longitudinal axis 201. In the illustrated embodiment, movable actuating element 208 is a bellows. The pump 206 is actuated by grasping the penile prosthesis near the bellows (just forward of the bellows) and pulling and pushing so as to contract and expand bellows 208. This creates a suction pumping force that draws the fluid from fluid reservoir 204 to enter and inflate inflatable tubular member 202.

One or more one-way valves 210, such as but not limited to, duckbill valves, may be provided on the distal side of pump 206, as well as the proximal side thereof. The one-way valve 210 allows fluid to enter and inflate inflatable tubular member 202 and prevents fluid from flowing back from inflatable tubular member 202 towards reservoir 204. In this manner the erection is maintained.

A deflation actuator 212 may be fluidly connected to the prosthesis, such as by tubes 214 on the distal and proximal sides of pump 206. The deflation actuator 212 may include a bulb which may be squeezed to force the fluid back from inflatable tubular member 202 towards reservoir 204 via the tubes 214, thereby bypassing the pump 206 and returning the penile prosthesis to a flaccid state. The deflation actuator 212 may include a valve that is closed during inflation and erection of inflatable tubular member 202 and open during deflation.

Reservoir 204 may include one or more stiffening members 216, such as but not limited to, a longitudinal stiffening rod that extends from the proximal end of reservoir 204 to the inlet to the pump 206.

Inflatable tubular member 202 may include one or more stiffening or protective members 218, such as but not limited to, silicone strings embedded in the material of the member 202 or a protective sheath around the member 202.

The penile prosthesis 200 may include proximal and distal end caps 220 and 222, respectively, which may be made of a polymeric or other suitable material.

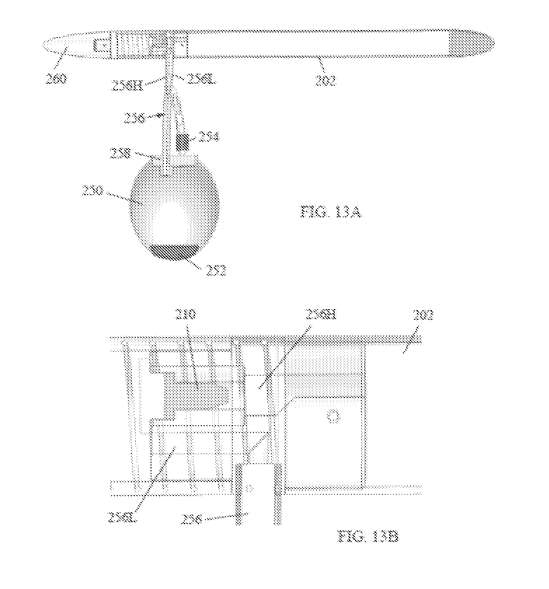

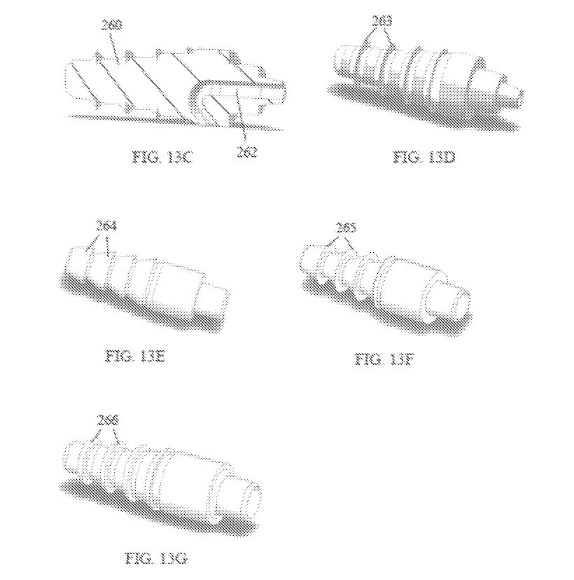

FIG. 13A shows another variation of the embodiment of FIG. 12. In FIG. 13A, there are no reservoir 204 and stiffening members 216. Instead, there is a reservoir 250 which initially contains the pumping fluid for inflating inflatable tubular member 202, and which also serves as the deflation actuator. Reservoir 250 may be initially filled by the surgeon during implantation of the prosthesis in the patient. After surgery, the physician or patient can add more fluid to the system via a filling septum port 252, which may be located at a bottom portion of reservoir 250 or other suitable location.

Reservoir 250 may include a discharge or safety valve 254. Valve 254 ensures that the pressure of the fluid does not exceed a preset value, such as but not limited to, 1.4-1.7 bar. If the fluid were to exceed the preset value, damage could occur to the prosthesis or cause injury or discomfort to the patient. Valve 254 ensures this does not happen. Valve 254 may be a 360° discharge valve, meaning the valve and its outlet may be oriented at any desired orientation.

Without limitation, the reservoir volume may be 100 ml if located in the abdomen or 40 ml if located in the scrotum. Reservoir 250 may be common for both the left and right prostheses which are located in the left and right corpus cavernosa.

Reservoir 250 may be fluidly connected to inflatable tubular member 202 by a multi-lumen tube 256. Multi-lumen tube 256 may be a kink-resistant tube with a high pressure lumen 256H and a low pressure lumen 256L, and connected to the reservoir and penile prosthesis with quick-connect fluid connectors. There is pressure equalization between the high pressure lumens of the left and right prostheses.

High pressure lumen 256H and low pressure lumen 256L may be located side-to-side or may be concentric with each other or may be one inside the other but non-concentric (centers of the lumens being offset from each other), or other possible arrangements, for example. The continuation of the fluid paths of high pressure lumen 256H and low pressure lumen 256L into the penile prosthesis are shown partially in FIG. 13B.

As described previously for the embodiment of FIG. 12, in the embodiment of FIG. 13A, the user may grasp the penile prosthesis and pull and push so as to contract and expand bellows 208 to create a suction pumping force that draws the fluid from fluid reservoir 250 to enter and inflate inflatable tubular member 202. Fluid flows from reservoir 250 through high pressure lumen 256H via a one-way valve 258 (e.g., a duckbill valve) located on reservoir 250.

As described previously for the embodiment of FIG. 12, in the embodiment of FIG. 13A, the bulb of reservoir 250 may be squeezed to force the fluid back from inflatable tubular member 202 towards reservoir 250 via low pressure lumen 256L to deflate inflatable tubular member 202.

In the embodiment of FIG. 13A, there is a proximal end cap 260 which may have different shapes as shown in FIGS. 13C-13G. As seen in FIG. 13C, proximal end cap 260 may include a fluid pathway 262, which may be part of the high pressure pathway or could also be part of the low pressure pathway. As seen in FIGS. 13D-13G, proximal end cap 260 may be tapered and include anchoring members to help anchor the prosthesis in the soft tissue of the patient. The anchoring members may include thin rings 263 (FIG. 13D), smooth conical members 264 (FIG. 13E), flared conical members 265 (FIG. 13F), or thick rings 266 (FIG. 13G). Other anchoring members may also be used.

Reference is now made to FIG. 13, which illustrates a penile prosthesis 300, in accordance with a non-limiting embodiment of the invention.

Similar to penile prosthesis 200, penile prosthesis 300 has a longitudinal axis 301 and includes an inflatable tubular member 302 which is more rigid when inflated than when deflated. Prosthesis 300 has a fluid reservoir 304 for holding fluid. Fluid reservoir 304 is in fluid communication with inflatable tubular member 302.

A pump 306 includes a movable actuating element 308 operative to transfer the fluid from fluid reservoir 304 to inflate inflatable tubular member 302 upon axial movement of the movable actuating element 308 along longitudinal axis 301. In the illustrated embodiment, movable actuating element 308 is a bellows. In all embodiments described herein, the bellows may be a corrugated sleeve member. The pump 306 is actuated by grasping the penile prosthesis near the bellows (just forward of the bellows) and pulling and pushing so as to contract and expand bellows 308. This creates a suction pumping force that draws the fluid from fluid reservoir 304 to enter and inflate inflatable tubular member 302. Pulling (distally extending) the bellows may provide gross filling, that is, the major part of inflating the inflatable tubular member 302. Secondary filling may be provided by pushing (proximally compressing) the bellows.

One or more one-way valves 310, such as but not limited to, duckbill valves, may be provided on the distal side of pump 306, as well as the proximal side thereof. The one-way valve 310 allows fluid to enter and inflate inflatable tubular member 302 and prevents fluid from flowing back from inflatable tubular member 302 towards reservoir 304. In this manner the erection is maintained.

A deflation actuator 312 may be fluidly connected to the prosthesis, such as by a one or more tubes 314 in fluid communication with inflatable tubular member 302 and reservoir 304, bypassing pump 306. The deflation actuator 312 may include a bulb which may be squeezed to force the fluid back from inflatable tubular member 302 towards reservoir 304 via tube or tubes 314 and return the penile prosthesis to a flaccid state. The deflation actuator 312 may include a valve that is closed during inflation and erection of inflatable tubular member 302 and open during deflation.

Reservoir 304 may include one or more stiffening members 316, such as but not limited to, a longitudinal stiffening rod that extends from the proximal end of reservoir 304 to the inlet to the pump 306. An additional reservoir may be provided if needed.

Inflatable tubular member 302 may include one or more stiffening or protective members 318, such as but not limited to, an inner sleeve (e.g., silicone sleeve), a middle sleeve (e.g., a layer of polyethylene terephthalate fabric) and an outer sleeve (e.g., silicone sleeve). These sleeves may improve implant durability and provide tensile strength during the pumping actuation.

A concentric tube stiffener 319, such as a plastic sheath, may extend from pump 206 towards the distal end of inflatable tubular member 302. The tube stiffener 319 is concentric with pump 306 and inflatable tubular member 302. The tube stiffener 319 may fit over a distal portion of pump 206. The tube stiffener 319 helps maintain concentricity of the pumping chamber with the rest of the prosthesis and also provides rigidity to the prosthesis.

The penile prosthesis 300 may include proximal and distal end caps 320 and 322, respectively, which may be made of a polymeric or other suitable material.

Each of the following terms: ‘includes’, ‘including’, ‘has’, ‘having’, ‘comprises’, and ‘comprising’, and, their linguistic/grammatical variants, derivatives, or/and conjugates, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of.’

Each of the phrases ‘consisting of’, and ‘consists of’, as used herein, means ‘including and limited to.’

The phrase ‘consisting essentially of’, as used herein, means that the stated entity or item (system, system unit, system sub-unit, device, assembly, sub-assembly, mechanism, structure, component, element, or, peripheral equipment, utility, accessory, or material, method or process, step or procedure, sub-step or sub-procedure), which is an entirety or part of an exemplary embodiment of the disclosed invention, or/and which is used for implementing an exemplary embodiment of the disclosed invention, may include at least one additional ‘feature or characteristic’ being a system unit, system sub-unit, device, assembly, sub-assembly, mechanism, structure, component, or element, or, peripheral equipment, utility, accessory, or material, step or procedure, sub-step or sub-procedure), but only if each such additional ‘feature or characteristic’ does not materially alter the basic novel and inventive characteristics or special technical features, of the claimed entity or item.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably. For example, for stating or describing the numerical range of room temperature, the phrase ‘room temperature refers to a temperature in a range of between about 20° C. and about 25° C.’, and is considered equivalent to, and meaning the same as, the phrase ‘room temperature refers to a temperature in a range of from about 20° C. to about 25° C.’

The term ‘about’, as used herein, refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments.

What is claimed is:

1. Apparatus comprising:
a penile prosthesis that has a longitudinal axis and which comprises an inflatable tubular member which is more rigid when inflated than when deflated;
a fluid reservoir for holding fluid and in fluid communication with said inflatable tubular member; and
a pump comprising a movable actuating element operative to transfer the fluid from said fluid reservoir to inflate said inflatable tubular member upon axial movement of said movable actuating element along said longitudinal axis.

2. The apparatus according to claim 1, wherein the axial movement of said movable actuating element comprises reciprocating movement alternatively towards proximal and distal portions of said penile prosthesis.

3. The apparatus according to claim 1, wherein said movable actuating element comprises a piston, piston rod or piston head.

4. The apparatus according to claim 1, wherein said penile prosthesis comprises a one-way valve through which said fluid flows towards said inflatable tubular member.

5. The apparatus according to claim 1, wherein said pump comprises a diaphragm connected to said movable actuating element.

6. The apparatus according to claim 4, wherein said diaphragm separates between a first chamber and a second chamber, and wherein said pump comprises at least one valve positioned within said first chamber and configured to allow fluid flow from said fluid reservoir towards said inflatable tubular member when pull forces are applied on said diaphragm in a direction towards a proximal portion of said penile prosthesis and to restrict fluid flow from said fluid reservoir when push forces are applied on said diaphragm in a direction towards a distal portion of said penile prosthesis.

7. The apparatus according to claim 1, wherein a tubing system is disposed on a periphery of the inflatable tubular member and fluidly connects between said fluid reservoir and said inflatable tubular member.

8. The apparatus according to claim 1, comprising a draining system configured to drain fluid from said inflatable tubular member back into said reservoir.

9. The apparatus according to claim 7, comprising a manifold configured to direct fluid flow from said tubing system to said inflatable tubular member.

10. The apparatus according to claim 9, wherein said manifold comprises at least one channel and a manifold body.

11. The apparatus according to claim 1, wherein said movable actuating element comprises bellows.

12. The apparatus according to claim 1, wherein said inflatable tubular member comprises one or more stiffening members.

13. The apparatus according to claim 1, wherein said reservoir comprises one or more stiffening members.

14. The apparatus according to claim 1, comprising a deflation actuator operative to force fluid back from said inflatable tubular member towards said reservoir via a tube that bypasses said pump.

15. The apparatus according to claim 1, comprising a tube stiffener concentric with said pump and said inflatable tubular member.
